(19) United States
(12) Patent Application Publication
(10) Pub. No.: US 2009/0192549 A1
(43) Pub. Date: Jul. 30, 2009

(54) BONE PLATING SYSTEM

(75) Inventors: Laurie SANDERS, Montclair, NJ (US); Gretchen DOUGHERTY SHAH, Wayne, NJ (US); Rui J. FERREIRA, Livingston, NJ (US)

Correspondence Address: HARNESS, DICKEY & PIERCE, P.L.C., P.O. BOX 828, BLOOMFIELD HILLS, MI, 48303, US

(73) Assignee: EBI, LLC, Parsippany, NJ (US)

(21) Appl. No.: 12/361,844

(22) Filed: Jan. 29, 2009

Related U.S. Application Data
(60) Provisional application No. 61/024,698, filed on Jan. 30, 2008.

(51) Int. Cl. A61B 17/70; A61B 17/58

(52) U.S. Cl. 606/280; 606/70; 606/301

(57) ABSTRACT

A bone plating system has a plate and a locking element. The plate has a bone screw aperture defined by a sidewall including first and second substantially planar segments. The locking element is coupled to the plate and at least partially positioned in the aperture. The locking element has an external geometry defining first and second substantially planar surfaces and an internal geometry for receiving a head of a bone fastener and preventing the bone fastener from backing out of the plate. The locking element is expandable from a first state to a second state. In the first state the locking element is permitted to articulate relative to the plate. In the second state the first and second substantially planar surfaces of the locking element engage the first and second substantially planar segments of the aperture sidewall, respectively, to prevent relative movement between the locking element and the plate.

### CROSS-REFERENCE TO RELATED APPLICATIONS

This application claims priority to U.S. Provisional Patent Application No. 61/024,698 filed 30 Jan. 2008, which application is herein expressly incorporated by reference.

### FIELD

The present teachings relate generally to orthopedic surgical procedures. More particularly, the present teachings relate to a system for fixating the cervical spine.

### INTRODUCTION

In certain orthopedic surgical procedures, it is necessary to secure multiple bones or bone portions relative to each other. For example, in certain spinal surgeries, the fusion of two or more vertebral bodies is required to secure a portion of the spinal column in a desired position. This need may be the result of physical trauma from fractures or dislocations, degenerative diseases, or tumors.

One such spinal fixation procedure involves the attachment of a prosthesis or plate to the anterior side of the cervical portion of the spine. The procedure requires anteriorly accessing the spine and securing a prosthetic plate to two or more cervical vertebrae. This allows fusion of the two or more cervical vertebrae in a particular orientation to facilitate healing or to alleviate a condition of a patient.

Various fusion plates and plating systems are known for anteriorly fusing the cervical spine. Examples of known plating systems are shown in commonly assigned U.S. Pat. No. 6,599,290, which is hereby incorporated by reference as if fully set forth herein. Such plates and plating systems must meet several requirements associated with spinal stability and system reliability over an extended period of use. Additionally, it may be further desirable to ensure that the bone fasteners placed into the bone through the plate do not loosen or back out from the plate. Furthermore, locking mechanisms for preventing loosening of the bone fasteners should adequately permit the removal of an associated bone fastener when required, and allow sufficient angular freedom for bone fasteners relative to a bone plate.

It remains desirable in the pertinent art to provide an improved bone plating system that addresses all the requirements discussed above.

### SUMMARY

In accordance with one aspect, the present teachings provide a bone plating system having a plate and a locking element. The plate has a bone screw aperture. The bone screw aperture is defined by a sidewall including first and second generally planar segments. The locking element is coupled to the plate and is at least partially positioned in the aperture. The locking element has an external geometry defining first and second generally planar surfaces and an internal geometry for receiving a head of a bone fastener and preventing the bone fastener from backing out of the plate. The locking element is expandable from a first state to a second state such that in the first state the locking element is permitted to articulate relative to the plate and in the second state the first and second substantially planar surfaces of the locking element engage the first and second substantially planar segments of the aperture sidewall, respectively, to prevent relative movement between the locking element and the plate.

In accordance with another aspect, the present teachings provide a bone plating system having a plate and a locking element. The plate has a bone screw aperture. The bone screw aperture is defined by a sidewall including first and second sidewall openings. The locking element is coupled to the plate and at least partially positioned in the aperture. The locking element has an internal geometry for receiving a head of a bone fastener and preventing the bone fastener from backing out of the plate and an external geometry defining first and second tabs extending into the first and second sidewall openings, respectively. The first and second tabs and sidewall openings are configured to normally allow articulation of the locking element relative to the plate in a first plane. At least one of the first and second tabs and at least one of the sidewall openings define cooperating stop surfaces for limiting a range of articulation in the first plane.

In accordance with yet another aspect, the present teachings provide a bone plating system having a plate with a bone screw aperture. The plating system additionally includes a constrained bone screw having a constrained head and a semi-constrained bone screw having a semi-constrained head. The plating system further includes a locking element at least partially positioned in the aperture and coupled to the plate for relative articulation within at least a first plane generally perpendicular to the plane of the plate. The locking element is configured to interchangeably receive both the constrained bone screw and the semi-constrained bone screw such that the respective constrained and semi-constrained heads are captured relative to the locking element for articulation with the locking element and prevented from backing out of the plate. The locking element is expandable from a first state to a second state such that in the first state the locking element is permitted to articulate relative to the plate and in the second state relative movement between the locking element and the plate is prevented. The constrained head is configured to expand the locking element to the second state when captured by the locking element. The locking element remains in the first state upon capture of the semi-constrained head.

In accordance with still yet another aspect, the present teachings provide a spinal plating system for securing a first vertebra and a second vertebra. The spinal plating system includes a plate and a locking element. The plate has first and second bone screw apertures for overlying the first vertebra and third and fourth bone screw apertures for overlying the second vertebra. At least one of the bone screw apertures is defined by a sidewall including first and second substantially planar segments. The first and second substantially planar segments include first and second sidewall openings, respectively. The locking element is at least partially positioned in the one of the apertures. The locking element has an external geometry defining first and second substantially planar surfaces and first and second tabs extending from the first and second substantially planar surfaces, respectively. The first and second tabs extend into the first and second sidewall openings, respectively. The locking element further includes an internal geometry for receiving a head of a bone fastener and preventing the bone fastener from backing out of the plate. The locking element is expandable from a first state to a second state such that in the first state the locking element is permitted to articulate relative to the plate and in the second state the first and second substantially planar surfaces of the locking element engage the first and second substantially planar segments of the aperture sidewall, respectively, to prevent relative movement between the locking element and the plate. At least one of the first and second tabs and at least one of the sidewall openings define cooperating stop surfaces for limiting a range of articulation in the sagittal plane.

Additional advantages and further areas of applicability of the present invention will become apparent from the following detailed description and appended claims. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 is a perspective view of a plating system according to the present teachings;

FIG. 2A is a top view of the plating system of FIG. 1 illustrated with bone screws removed for purposes of illustration;

FIG. 2B is an end view of the plating system of FIG. 2A;

FIG. 2C is a side view of the plating system of FIG. 2A;

FIG. 2D is a cross-sectional view taken along the line 2D-2D of FIG. 2A with a locking member seated within the plating system according to the present teachings;

FIG. 2E is a partially cut away view of a portion of the plating system of FIG. 2A.

FIG. 3A is a cross-sectional view similar to FIG. 2D illustrated with bone fasteners seated within the plating system as implanted;

FIGS. 3B through 3D represent a series of views similar to FIG. 3A illustrating the bone fasteners during implantation;

FIGS. 4A through 4E are various views of locking members in accordance with the present teachings;

FIGS. 5A through 5C are various views of the locking member of FIG. 4A;

FIG. 6A is an enlarged and simplified side view of the detail of FIG. 2A operatively illustrated with the bone screw;

FIG. 6B is a view similar to FIG. 6A, the bone fastener shown articulated from the orientation of FIG. 6A;

FIG. 7A is a cross-sectional view taken through another locking member in accordance with the present teachings;

FIG. 7B is a view similar to FIG. 6B illustrating the locking member of FIG. 7A;

FIG. 8 is a view similar to FIG. 5C illustrating an alternative tab geometry;

FIGS. 9A through 9F are cross-sectional views of additional locking elements in accordance with the present teachings; and

FIGS. 10A and 10B are top views of a portion of an alternative plate in accordance with the present teachings, the plate shown operatively associated with the locking element with the locking element in a first state in FIG. 10A and in a second state in FIG. 10B.

### DESCRIPTION OF VARIOUS ASPECTS

The following description is merely exemplary in nature and is in no way intended to limit the invention, its application, or uses. For example, although the present teachings are illustrated for internal fixation of the cervical spine, the present teachings can also be used for other orthopedic procedures in which it is necessary to secure two bone portions relative to one another.

With general reference to the drawings, various bone plating systems are illustrated. The bone plating systems will be understood to generally include a bone plate and bone fasteners or screws for securing the plate to bone. Additionally, the bone plating systems include locking elements or mechanisms for locking the bone fasteners relative to the respective plate. For ease of reference and despite differences between the various embodiments, reference character 10 has been used throughout the drawings to generally identify the various bone plating systems of the present teachings. Similarly, the bone plates are identified throughout with reference character 12, the locking elements are identified throughout with reference character 14, and the bone fasteners are identified throughout with reference character 16. The differences between the various embodiments will be addressed below and are further shown in the drawings.

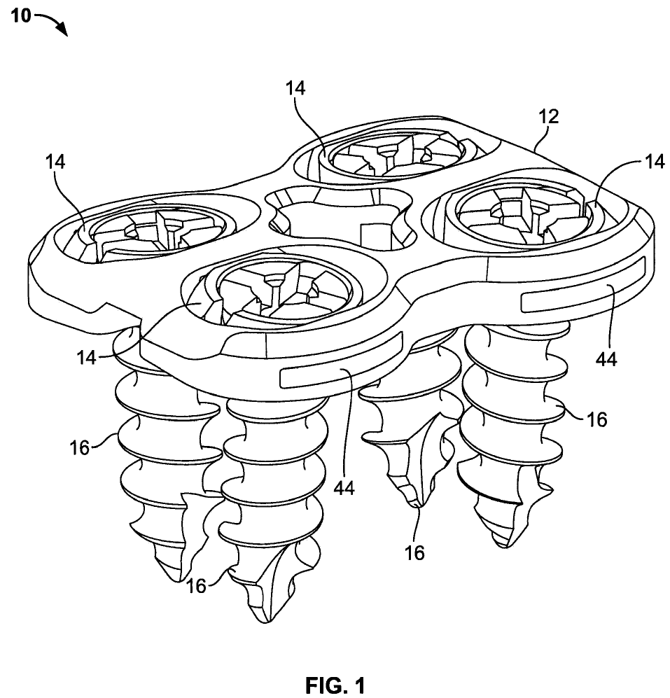

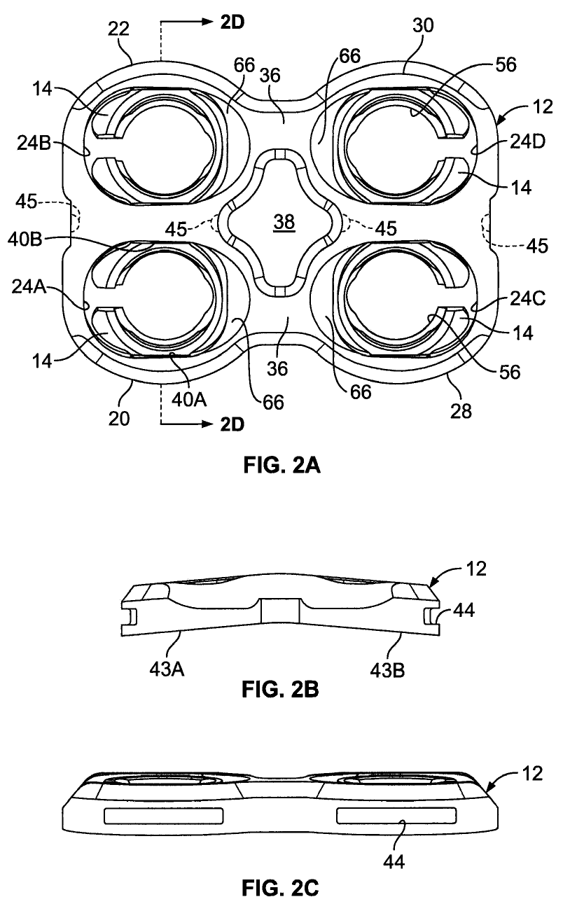

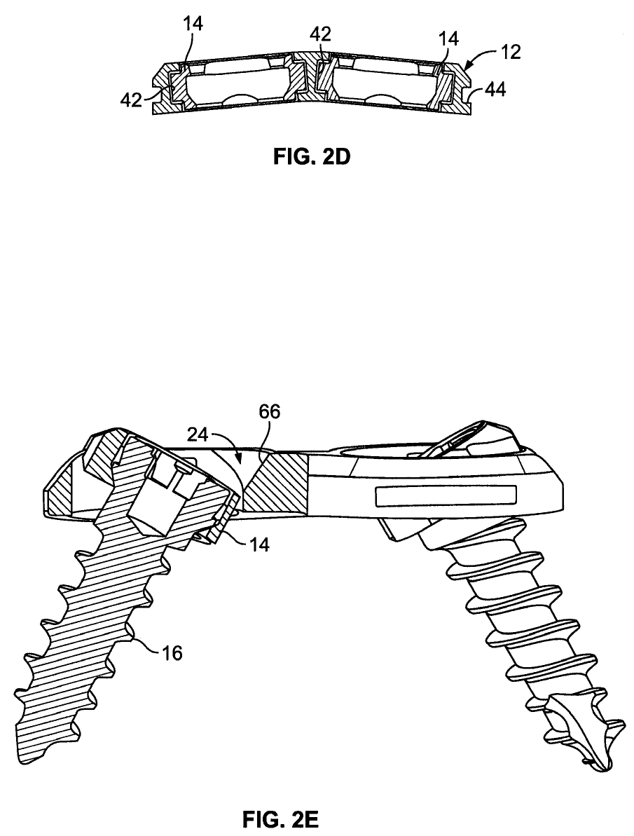

With reference to FIGS. 1 and 2A through 2D, the bone plating system 10 having the bone plate 12 particularly adapted for a one-level spinal fixation is illustrated. Bone plates for additional levels may be similarly constructed in accordance with the present teachings. In certain applications, the bone plates 12 may have a maximum thickness of approximately 2.5 mm and a maximum width of approximately 16.5 mm.

The bone plate or plate member 12 includes a first pair of nodes having a first nodule 20 and a second nodule 22. The first and second nodules 20 and 22 define first and second plate holes or bone screw apertures 24A and 24B, respectively. The first and second nodules 20 and 22 are generally laterally spaced apart from one another in a first direction. In the embodiment illustrated and in a manner to be more fully discussed below, the first and second bone screw apertures 24A and 24B are intended to receive the fasteners 16 for engaging a first vertebral body. In a similar manner, the plate member 12 includes a second pair of nodes having a third nodule 28 and a fourth nodule 30. The third and fourth nodules 28 and 30 define third and fourth plate holes or bone screw apertures 24C and 24D, respectively. Again, the third and fourth nodules 28 and 30 are generally spaced apart from one another in a lateral direction. The third and fourth bone screw apertures 24C and 24D are intended to receive the fasteners 16 for engaging a second vertebral body.

The plate member 12 is further shown to include a plurality of linking segments 36 which connect the first and second pairs of nodes. The linking segments 36 extend in a longitudinal or axial direction which is essentially perpendicular to the lateral direction in which the nodules 20, 22 and 28, 30 of the first and second pairs of nodes are spaced apart. The linking segments 36 define a viewing window 38. Explaining further, the viewing window 38 may permit intra-operative visualization of a bone graft, as well as post-operative visualization of bone graft consolidation and spinal orientation on an anterior/posterior x-ray.

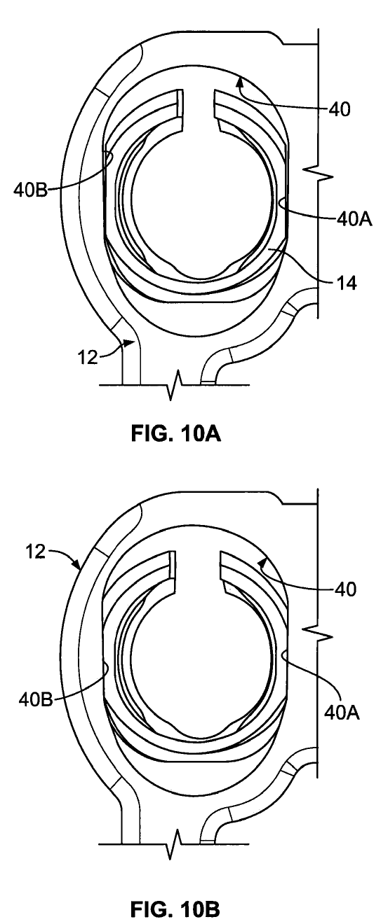

As shown in the top view of FIG. 2A, the bone screw apertures 24 may be generally rectangular with rounded corners and may be elongated along the axis of the plate 12. As used herein, the term “generally rectangular” shall be interpreted to include quadrilaterals, trapezoids, and similar shapes. In particular, the apertures 24 may be bounded by a sidewall having a pair of generally planar segments 40A and 40B. The generally planar segments 40A and 40B may be laterally spaced apart and generally parallel to one another. As used herein, the term “generally parallel” shall include the relationships shown in FIGS. 2A and 10A and similar relationships for accomplishing the same objective, including but not limited to segments that taper relative to one another (as discussed below). As shown in FIG. 2D, for example, the sidewall segments 40A and 40B may define sidewall openings 42 that cooperate with the locking element 14 to retain the locking element 14 to the plate 12. The sidewall openings 42 may be generally rectangular.

As particularly shown in FIGS. 2B and 2D, the plate member 12 may be contoured about a longitudinally extending midline through the viewing window 38. In this regard, the plate member 12 is shown to include a first lateral half 43A oriented at an obtuse angle relative to a second lateral half 43B. In one application, the obtuse angle is between approximately 160 degrees and 170 degrees. While not particularly shown in the drawings, the plate member 12 may additionally or alternatively be contoured to include a lordotic angle. Such a lordotic contour of the plate member 12 may reduce or eliminate manual fashioning of the plate member 12 to fit the contour of the spine, thereby decreasing surgical time. The contour of the plate member 12 may also decrease interference with adjacent soft tissue after implantation.

The plate 12 may be provided with chamfered cephalad and caudal edges. The chamfered edges may provide a smooth finish to the edges of the plate 12. The chamfered edges may decrease the chances of dysphasia and may also give a sleek lateral X-ray post-operatively. The chamfered cephalad and caudal edges are shown particularly in FIG. 2C. The chamfered medial/lateral edges are shown particularly in FIG. 2B.

The plate 12 may be provided with instrument fixation grooves 44. The instrument fixation grooves 44 may be cut along the medial/lateral edges of the plate 12 and may allow for fixation of instrumentation which seats into the grooves 44 without resting underneath the plate 12. In this manner, the instrumentation will not induce lifting of the plate 12 from a vertebral body. Instrumentation fixation undercuts may additionally be provided in the form of pockets on the underside of the plate 12 that are located at the cephalad and caudal ends and at the edges of graft visualization windows near fastener holes.

As particularly shown in dotted lines in FIG. 2A, the underside of the plate 12 may include notches or recesses 45 to accommodate a grasping tool. In this regard, the plate 12 may be grasped without lifting from the adjacent vertebrae. As shown, the recesses 45 may be formed adjacent the viewing window 38 and adjacent the edges of the plate 12 along a longitudinal centerline.

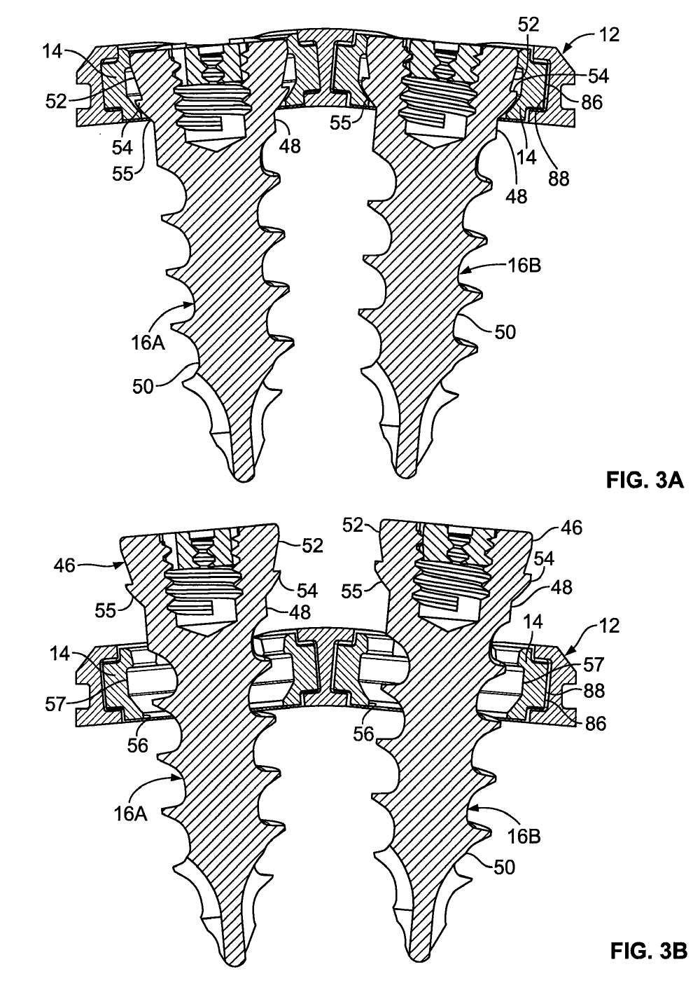

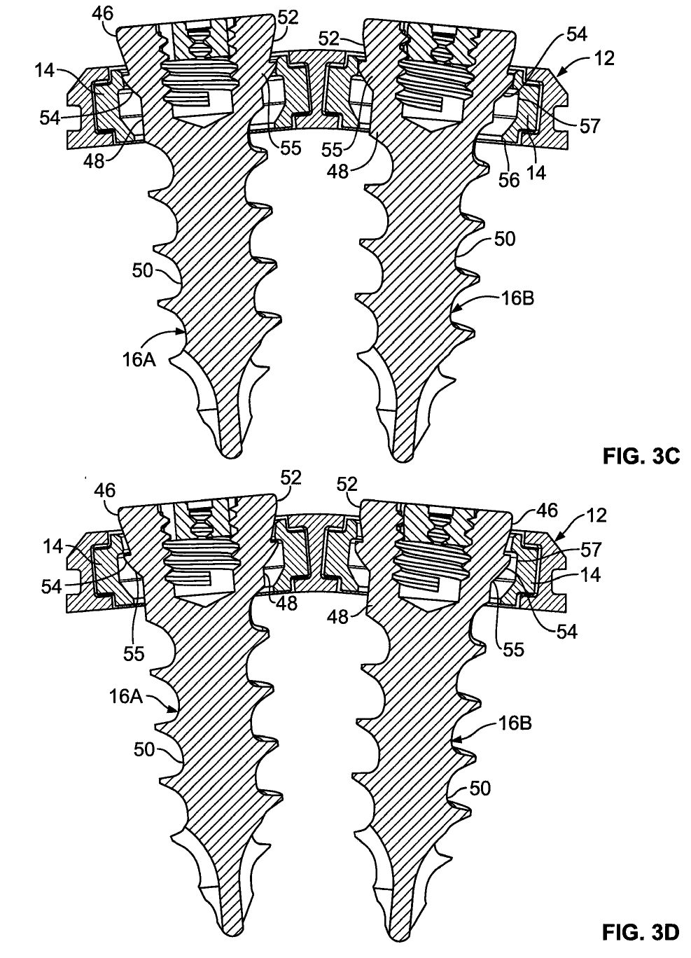

Referring now to FIGS. 3A through 3D, the bone plating system 10 of the present teachings may include first and second types of bone fasteners or bone screws 16A and 16B. The bone screw apertures 24 and the locking elements 14 may be adapted to interchangeably receive both the first and second types of fasteners 16A and 16B. In the embodiment illustrated, the first type of bone fastener may be a fixed-angle or constrained fastener 16A and the second type of bone fastener may be a variable-angle or semi-constrained fastener 16B. The fixed-angle fasteners 16A may cooperate with the plate member 12 and the respective locking element 14 to restrict or prevent relative movement. As will be discussed in further detail below, the variable-angle fastener 16B may cooperate with the plate member 12 and the respective locking element 14 to provide a range of articulation for the bone fasteners 16 relative to the plate 12. Such relative articulation may allow for operative freedom in obtaining purchase of the bone fastener 16 in the vertebral bodies or other bone. Both fasteners 16A and 16B may be placed at various angels relative to the plate 12. The fixed angle fasteners 16A may cooperate with the plate member 12 and a respective locking member 14 to restrict or prevent relative movement upon implantation.

As illustrated, each of the bone fasteners 16A and 16B may generally include a head or head portion 46, a neck portion 48 and a shaft portion 50. The head portion 46 may include an upper portion 52 that tapers as it extends downwardly and a lower, circumferentially extending lip 54. The lip 54 may have a tapered lower surface 55. The upper portion 52 may be configured to cooperate with one or more insertion/removal tools in any manner well known in the art. The specific features of the shaft portion 50, such as thread pitch, shaft diameter, and the like, are a matter of design choice and surgical preference. The diameter of the lip 54 of the bone fastener 16 may be larger than an opening 56 of the locking element 14, thereby preventing the bone fastener 16 from passing completely through the locking element 14 and capturing the head portion 46 of the fastener 16.

The locking element 14 may be at least partially positioned in the respective aperture 24. In this regard, the locking element 14 may sit proud of the aperture 24 or may extend partially from the aperture 24 through its range of articulation. The locking element 14 may be in the form of a locking ring. More particularly, the locking element 14 may be a split locking ring and may be retained within the aperture 24.

As such, discrete fasteners or other locking mechanisms may be eliminated. As will be addressed further below, the locking element 14 may be expanded from a first state (see FIG. 3B, for example) to a second state (see the locking element 14 on the left of FIG. 3A, for example) such that in the first state the locking element 14 is permitted to articulate relative to the plate 12 and in the second state relative movement between the locking element 14 and the plate 12 is prevented. As used herein, the term “first state” shall be interpreted to include any orientation, static or partially expanded, in which relative movement between the locking element 14 and the plate 12 is permitted. The term “second state” shall be interpreted to include an expanded locking element orientation in which relative movement is prevented.

As noted above, the outer geometry of the locking element 14 may be generally oval and the bone screw apertures 24 may be generally rectangular with rounded edges. Alternatively, the outer diameter of the locking element 14 may be circular, square or of any other suitable shape. The opening or thru-hole 56 in the locking element 14 may be generally circular. In certain applications, a central axis of the locking element thru-hole 56 may be offset approximately 0.010 in (0.25 mm) from a central axis of the locking element 14. As such, when the locking element 14 is placed in the aperture 24 in the plate 12, the central axis of the locking element 14 and the central axis of the aperture 24 may be offset approximately 0.005 in (0.125 mm). Resultantly, the central axis of the thru-hole 56 in the locking element 14 and the central axis of the aperture 24 may be offset approximately 0.015 in (0.375 mm). Alternatively, the respective axes may be aligned.

The inner geometry of the locking element 14 may define an enlarged and generally circular opening 57 for receiving at least a portion of the head 46 of the bone fastener 16. As will be addressed further below, the outer periphery of both the upper portion 52 of the head 46 and the lip 54 may engage the inner geometry of the locking element 14 in line contact such that the bone fastener 16 and the locking element 14 will articulate together relative to the plate 12.

In order for the locking elements 14 to capture the associated bone fasteners 16, the locking elements 14 may elastically flex or spring open and shut. In this regard, the locking element 14 may flex open to create a friction lock at the fastener/locking element and locking element/plate interfaces.

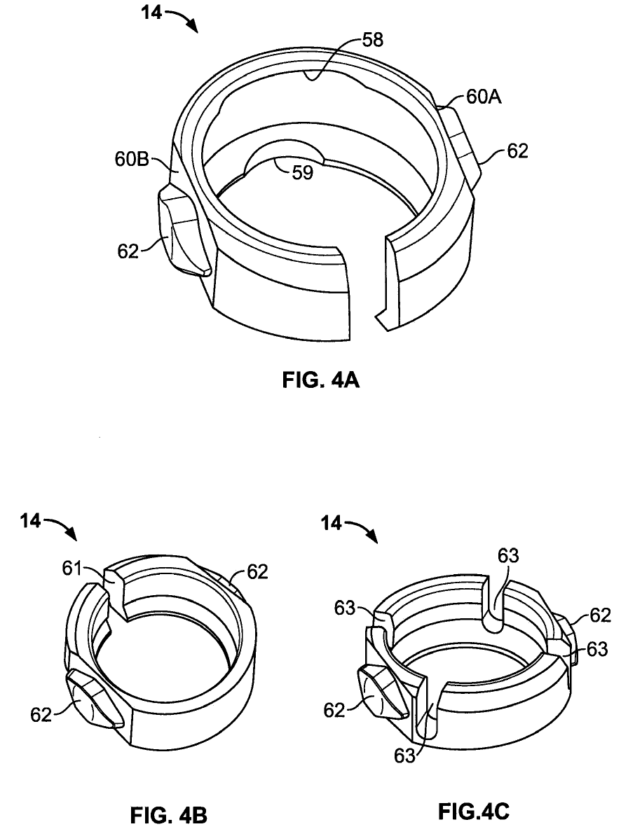

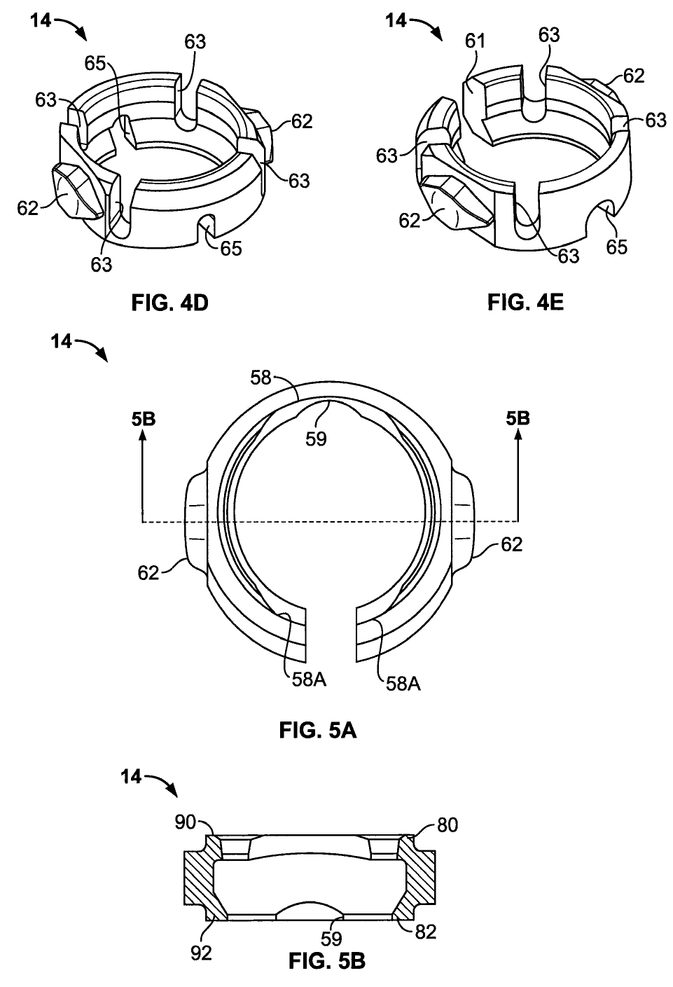

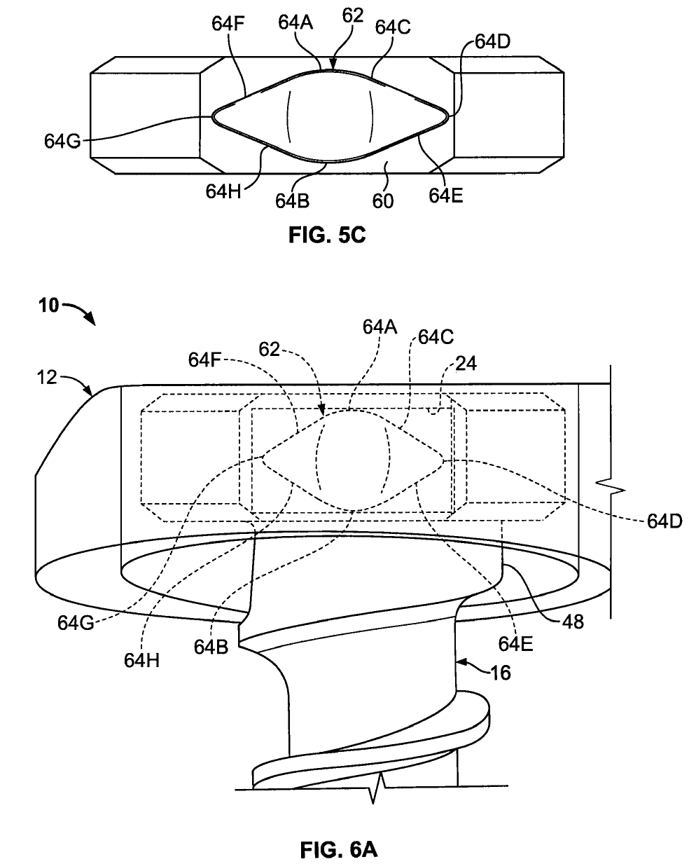

With reference to FIGS. 4A and 5A, different features (e.g., slots, grooves, undercuts, etc.) may be incorporated into the locking element 14 to decrease the rigidity of the locking element 14 and thereby facilitate elastic flexion of the locking element 14. For example, the locking element 14 is illustrated including a cooperating groove arrangement. A first large sweeping groove 58 is provided on an upper lip of the locking element 14 in one or multiple locations. Similar sweeping grooves 58A (see FIG. 5A) may be provided opposite the groove 58. A second sweeping groove 59 is provided on a bottom lip of the locking element 14 in one or multiple locations. Both the first groove 58 and the second groove 59 may help to decrease the rigidity of the locking element 14 at the main point of flexion. With this geometry, the locking element 14 may more easily spring open and shut to accept the bone fasteners 16 and lock them to the plate 12 in the manner discussed herein. In addition, material removed at the upper lip of the locking element 14 (e.g. with the first sweeping groove 58) may also help to decrease surface area contact between the locking elements 14 and the bone fasteners 16. This reduction in surface area contact may facilitate passing of the bone fasteners 16A into the locking element 14 with less resistance.

FIGS. 4B through 4E illustrate additional examples of ways to decrease the rigidity of the locking element 14. Consistent throughout these examples is removal of material from the top and bottom lip of the locking element 14. Some of the locking elements 14 include a slotted feature 61 that allows the locking element 14 to spring open by placing most of the stress on the locking element 14 at the portion opposite the slotted feature 61. This geometry causes the side opposite the slotted feature 61 on the locking element 14 to act slightly like a hinge. The locking elements 14 that do not include the slotted feature 61, may spring open slightly more uniformly by displacing outwardly from the central axis of the opening 56.

In particular, the embodiment of FIG. 4B illustrates the single slotted feature 61. The embodiment of FIG. 4C illustrates four top slots 63 extending partially through the locking element 14. The embodiment of FIG. 4D illustrates the four top slots 63 and two bottom slots 65 extending partially through the locking element 14. The embodiment of FIG. 4E incorporates all of these features including the partial slots on the top 63 and the bottom 65 and the slotted feature 61. As can be seen in the figures, the particular dimensions of the slotted features may vary. Additional combinations of the features of these embodiments are also anticipated within the scope of the present teachings.

With continued reference to FIG. 4A and further reference to FIGS. 5A through 5C, the exterior geometry of the locking element 14 may be configured to cooperate with the geometry of the aperture 24 for selectively preventing relative movement between the locking element 14 and the plate 12. For example, the locking element 14 may include external geometry defining first and second substantially planar surfaces 60A and 60B. When the locking element 14 is expanded with the constrained screw 16A from the first state (e.g., left locking element 14 in FIG. 3B or 3D) to the second state (e.g. FIG. 3A), the first and second generally planar surfaces 60A and 60B of the locking element 14 may engage the generally planar segments 40A and 40B of the sidewall 40 to prevent relative movement between the locking element 14 and the plate 12.

The exterior geometry of the locking element 14 may be further configured to couple the locking element 14 to the plate 12. In certain applications, the exterior geometry may be configured to define the range of permissible movement of the locking element 14 relative to the plate 12. In this regard, the exterior geometry of the locking element 14 may include a pair of tabs 62 for coupling the locking element 14 to the plate 12 and defining the range of movement between the locking element 14 and the plate 12.

The tabs 62 on the locking element 14 may be disposed in the sidewall openings 42. In certain applications, the sidewall openings 42 may be formed as undercuts within the plate 12. The sidewall openings 42 may be positioned at laterally opposite sides of the aperture 24. The tabs 62 and sidewall openings 42 may cooperate to define the relative movement normally permitted between the locking element 14 and the respective aperture 24.

The sidewall openings 42 and associated tabs 62 on the locking element 14 may be any suitable shape and any suitable size. The shapes and sizes may be modified to increase or decrease angulation of the locking element 14 (and in turn the associated bone fastener 16) in the cephalad/caudal direction. Such increased angulation may be facilitated through modification of the tabs 62 and/or the plate 12 ensuring that the plate 12 (1) does not interfere with the angulation of the locking element 14; and (2) captures the tabs 62 in a way that allows the locking element 14 to maximize its angulation in the cephalad/caudal direction.

With particular reference to FIGS. 2A and 2E, the aperture 24 may be formed to include a recess 66 for fastener insertion at an extreme angle. The recess 66 may be defined at a first circumferential portion of the aperture sidewall. The recess 66 may extend completely through the plate 12 and may be in the form of a cutout in the plate 12 that allows for the clearance of the fastener 16 and associate tools (not shown) during insertion of the fastener 16 at extreme angles without interference with the plate 12. This additional clearance may allow for the fastener 16 to be inserted at a steeper angle than would normally be permissible due to the geometry of the plate 12 without unnecessarily comprising the performance or strength of the plate 12. The recesses 66 may provide the noted advantages for systems both including and excluding locking elements 14.

As illustrated, the recess 66 may define a portion of a generally cylindrical shape. In the embodiment illustrated, the recess 66 provides for extreme angulation in one of the caudal or cephalad directions. Additional similar recesses may be defined to provide for extreme angulation in more than one direction. Further in the embodiment illustrated, in which the fastener 16 may angulate relative to the plate 12 approximately 30 degrees, the cylinder may extend at an angle of at least about 30 degrees relative to an axis of the aperture. It will be understood that the aperture axis is generally perpendicular to the plane of the plate 12. In certain applications, the cylinder may extend at an angle of at least approximately 25 degrees to the aperture axis. In particular applications, the cylinder may extend at an angle of at least approximately 30 degrees relative to the aperture axis. In embodiment illustrated, cylinder may extend at an angle of approximately 35 relative to the aperture axis.

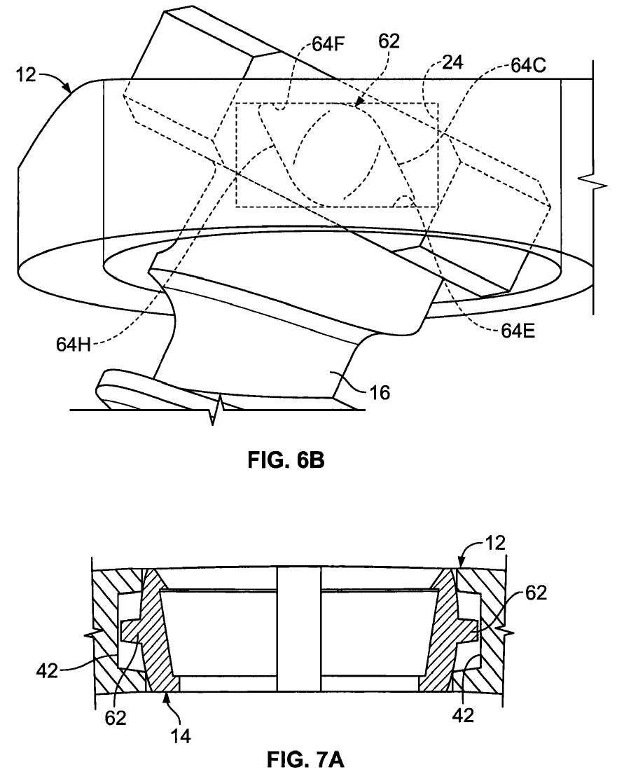

As illustrated most clearly in FIGS. 5C and 6A, the tabs 62 may include a plurality of sides 64. In the embodiment illustrated, the tabs include six sides 64 and the tab may have a shape generally like a football. A first or uppermost side 64A and a second or lowermost side 64B may define arcs lying on a common circle. Alternatively, the first and second sides 64A and B may be generally planar. Third, fourth, fifth and sixth sides 64C, 64E, 64F and 64H may be generally planar. The third side 64C may extend downwardly from the first side 64A at a tangent to the first side 64A to a first nose 64D. The fourth side 64E may upwardly extend from the second side 64B at a tangent to the first side 64A to the first nose 64D. Similarly, the fifth side 64F may downwardly extend from the first side 64A at a tangent to the second side 64B to a second nose 64G and the sixth side 64H may upwardly extend from the second side 64B at a tangent to the second side 64B to the second nose 64G. This geometry may provide the tabs 62 with increased strength without compromising the angulation of the locking element 14 relative to the plate 12.

FIG. 6A illustrates the tab 62 when the locking element 14 and fastener 16 are at neutral positions of 0 degrees. This neutral position is defined as the point when the shaft 50 of the bone fastener 16 is normal to the bottom of the plate 12. FIG. 6B illustrates the tab 62 when the fastener is at its extreme cephalad/caudal angulation. It should be noted that the fastener 16 may similarly articulate in the opposite direction. In the extreme angulation orientation, the flat faces 64E and 64F of the football shaped tab 62 are parallel to the faces defining the upper and lower boundaries of the respective sidewall opening 42.

The shape and size of the tabs 62 may be determined by choosing a diameter equivalent to or smaller than the height of the sidewall opening 42 and connecting straight lines tangent to that circle at a predetermined angle which mirrors the desired extreme angulation requirements. If the original circle is neglected and the straight lines are connected to each other, a rhombus is formed. This rhombus creates a large surface area so that when the locking element 14 is angulated to its most extreme cephalad/caudal position, the edges of the rhombus bottom out on the upper and lower lips of the sidewall opening.

In the embodiment shown particularly in FIGS. 6A and 6B, the tab 62 and the sidewall opening 42 are configured to allow articulation in a first or saggital plane. This articulation may be between approximately 20 degrees and approximately 35 degrees in both the cephalad and caudal directions. In the embodiment illustrated, the articulation may be at least approximately 30 degrees in both the cephalad and caudal directions. In other embodiments, the angulation may range from 0° to a range unrestricted by the tabs 62.

The tab 62 and the sidewall opening 42 may define cooperating stop surfaces for limiting the range of articulation in the first plane. When the locking element 14 is articulated fully in a first direction (FIG. 6B), the fifth side 64F may contact an upper surface of the sidewall opening 42 and the fourth side 64E may contact a lower surface of the sidewall opening 42. When the locking element 14 is rotated fully in the opposite direction, the third side 64C may contact the upper surface of the sidewall opening 42 and the sixth side 64H may contact the lower surface of the sidewall opening 42.

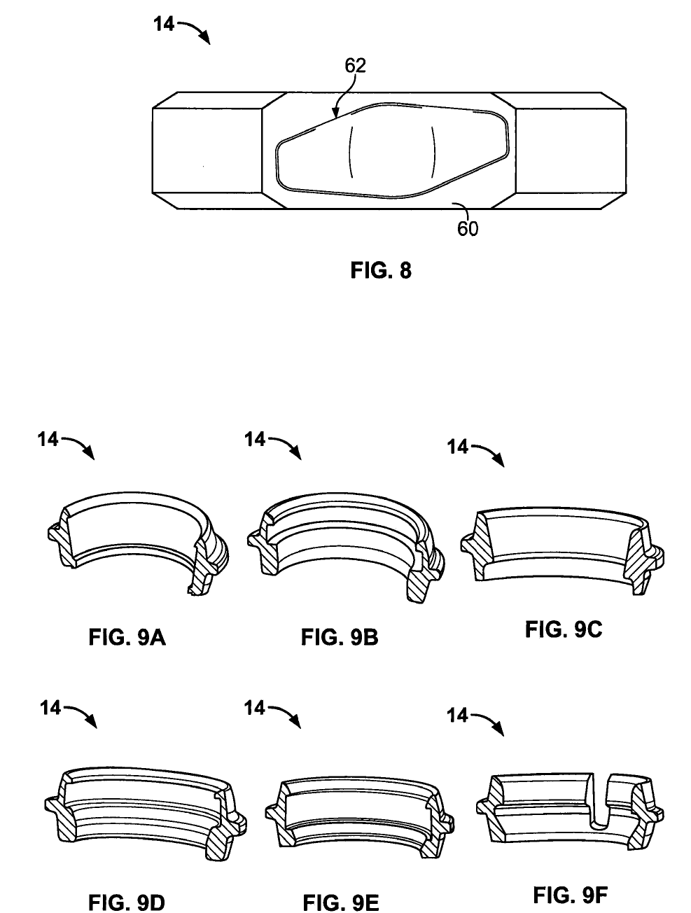

Turning to FIG. 8, a simplified view of a modified tab geometry is illustrated. In certain applications, it may be desired to further limit the angulation of the locking element 14 so that increased angulation can be obtained in only one direction (e.g., cephalad or caudal). For such applications, the relative geometries of the tab 62 and sidewall opening 24 may be modified. For example, the tab 62 may be modified as shown in FIG. 8 so that relative motion is only clockwise (as illustrated). This modified geometry may be hexagonal and can only be angled in one direction (i.e., counterclockwise as shown in FIG. 8) when placed in the sidewall opening 42. In this manner, the size of the tabs 62 may be maximized to help ensure that the tabs 62 will remain in the sidewall opening 42. Alternatively, the shape of the sidewall opening 24 may be modified to achieve the same objective.

As shown particularly in FIG. 6A, the distance between the first and second noses 64D and 64G may be smaller than the length of the sidewall opening 42. As such, the tabs 62 may translate within the sidewall opening 42. In turn, the locking element 14 and the fastener 16 may normally translate relative to the plate 12 in the same plane. In certain applications, the translation may be about 2.0 mm. Further, as the height of the tabs 62 is substantially equal to the height of the sidewall opening 42, articulation of the locking element 14 in a plane perpendicular to the plate plane is substantially prevented. In the application illustrated, the second plane is the anatomical transverse plane.

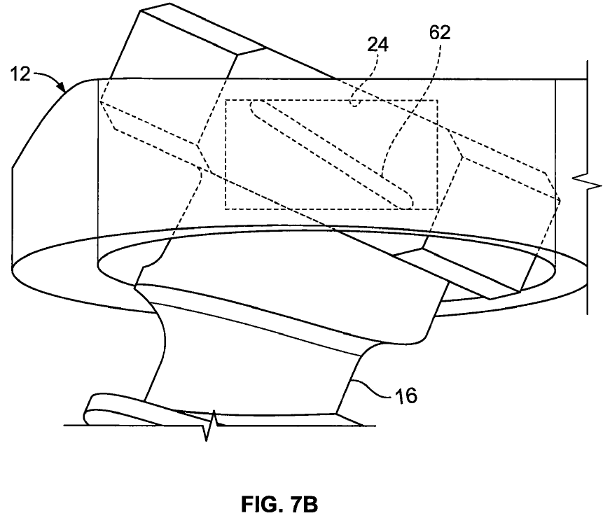

The cooperating geometries of the tabs 62 and the sidewall opening 42 may be modified to normally allow articulation of the locking element 14 relative to the plate 12 in the second plane. With reference to FIGS. 7A and 7B, such a modified relationship is illustrated. Here, the height of the tab 62 is significantly less than the height of the sidewall opening 42. The range of angulation in the first plane may be significantly greater than the range of articulation in the second plane. In certain applications, the range of articulation in the second plane may be limited to approximately ±5 degrees.

In the embodiments illustrated throughout the drawings, the various locking elements 14 are adapted to interchangeably receive both fixed-angle bone fasteners 16A and variable-angle bone fasteners 16B. Alternatively, the internal geometry of the locking element 14 may vary in accordance with whether the locking element 14 is to be used with the constrained or fixed-angle bone fasteners 16A or with the semi-constrained or variable-angle bone fasteners 16B. When the locking elements 14 are paired with the corresponding bone fasteners 16A, 16B: (1) a mechanical lock is created such that the bone fastener 16A, 16B cannot back out of the construct in vivo; and (2) the bone fastener 16A, 16B may be inserted at any angle within a predetermined range. As discussed further below, the internal geometries may utilize a taper that can be used to splay the locking element 14 open to work with the fixed-angle bone fastener 16A, or an undercut that can work to capture the variable-angle bone fastener 16B.

With particular reference to FIG. 5, the locking element 14 may include an upper tapered portion 80 and a lower tapered portion 82. The generally tapered portions may involve a gentle curve, or a spherical or conical geometry. The upper tapered portion 80 may be relatively smaller than the lower tapered portion 82. The lower tapered portion 82 may include the recess 59 and a spherical pocket that sits at the bottom of the bone screw aperture 24. The two tapers 80, 82 may cooperate to help form the mechanical lock for both styles of bone fastener 16A, 16B. It will be noted that the undercut or recess 59 may be in any shape or geometry to assist in accomplishing this objective.

The cooperative action between the bone fasteners 16 and the locking elements 14 will be described in further detail with reference back to the cross-sectional views of FIGS. 3A through 3D. As the bone fastener 16 begins to pass through the locking element 14 (FIG. 3C), the upper tapered portion 80 may deflect radially outward to allow the bone fastener 16 to pass into the opening 56. When the lip 54 of the fastener 16 passes the upper tapered portion 80, the head 46 of the fastener 16 is captured within the locking element 14. While both fasteners 16A and 16B are described to include a mechanical lock to prevent backout, the constrained fastener 16A may alternatively be retained relative to the locking element 14 (and thereby the plate 12) solely through a frictional interference.

Upon seating the head 46 of the constrained fastener 16A (see FIG. 3A), the upper tapered portion 80 may splay open the locking element 14 and rigidly fix the locking element 14 to the plate 12 by friction forming a constrained construct (e.g., preventing relative movement between the locking element 14 and the plate 12). Frictional interference may be established between the generally planar surfaces 60A and 60B of the locking element 14 and the generally planar segments 40A and 40B of the sidewall 40, respectively. Alternatively, outward faces 86 of the tabs 62 may define the generally planar surfaces of the locking element 14. These outward faces 86 may frictionally engage an inner surface 88 of the bone plate 12 and define the cooperating generally planar segments of the aperture 24. In certain applications, locking of the locking element 14 relative to the plate 12 may be accomplished with a hybrid of the arrangements discussed herein.

The semi-constrained fastener 16B may be arranged similarly to the fixed fastener 16A in that it may have the upper tapered portion 80 utilized in combination with the inner geometry of the locking element 14 to create a mechanical lock for precluding back-out of the fastener 16B. However, the semi-constrained fastener 16B does not rely on frictional interference between the locking element 14 and the fastener 16B. Instead, the upper portion of the head 46 of the semi-constrained fastener 16B is slightly undersized and the lip 54 of the fastener 16B snapping into the undercut 84 activates the mechanical lock. In this way, the locking element 14 is not splayed open to thereby cause it to lock by friction to the plate 12. Instead, the fastener 16B remains variable or semi-constrained, which allows for micro-motion and settling upon graft subsidence.

Similarly, both the constrained and semi-constrained fasteners 16A and 16B may be removed from the locking element 14 by applying pressure to a top surface 90 of the locking element 14 while the fastener 16 is forced upward. Due to the small amount of interference between the fastener 16 and the upper tapered portion 80 of the locking element 14, the fastener 16 itself may assist to reverse the locking process by splaying the locking element 14 open when the fastener 16 is forced upwards and the locking element 14 is held down. These cooperating geometries may provide for easy removal and rework when necessary.

Alternate designs for the upper tapered portion 80, the lower tapered portion 82, and the undercut 84 are shown in FIGS. 9A through 9F. Similar to the embodiment of FIG. 5, mechanical locking may occur when coupled to their respective fixed- or variable-angle bone fasteners 16A and 16B. It should be noted that the embodiments shown in FIGS. 9B, 9C, 9D, and 9E have a true mechanical lock for the variable-angle bone fasteners 16B, but not for the fixed-angle fasteners 16A. Instead, these designs rely on friction between the fastener/locking element/plate interface to prevent back-out.

The embodiment of FIG. 9A illustrates a central taper/undercut. The embodiment of FIG. 9B illustrates a continuous upper/lower taper with cyclindrical undercut. The embodiment of FIG. 9C illustrates a taper with bottom ring undercut. The embodiment of FIG. 9D illustrates a lower taper angle with cylindrical undercut. The embodiment of FIG. 9E illustrates a double taper angle on both upper and lower locking element lips. The embodiment of FIG. 9F illustrates an upper taper with spherical undercut. These features may be used alone or in various combinations.

With reference to the various figures and particular reference to FIGS. 10A and 10B, the plate 12 may incorporate apertures 24 define a generally trapezoidal shape including a taper. In this regard, the planar segments 40A and 40B of the sidewall 40 may slightly approach one another in a direction away from the slotted feature 61 of the locking element 14. Such a geometry may be desirable when incorporating the slotted feature 61 due to the hinge-like nature of the locking element 14 when it is expanded with the constrained fastener 16A. When the locking element 14 is expanded by the constrained fastener 16A, the parallel edges 60A, 60B that make up the locking element 14 at its relaxed state splay out at an angle between approximately 1 and 10 degrees. Therefore, when between approximately 1 and 10 degrees of motion is applied to the aperture 24 in the plate 12, the locking element 14 splays open the edge of the locking element 14 to match up with the edges of the plate 12. In this manner, the locking element 14 is securely fixed to the plate 12 though friction due to the increased surface area contact between the two mating parts.

In addition to the increased security of the construct due to increased surface area contact between components, this type of configuration may also make the construct stronger in compression. When the construct is under a compressive load, the bone fastener 16 desires to sweep in the cephalad or caudal direction towards the center of the plate 12. By having the aperture 24 trapezoidal in shape, the locking element 14 prevents the fastener 16 from moving in the sweeping motion by forcing the locking element 14 deeper into the wedge created by the angled trapezoidal geometry.

The described geometrical relationship between the locking element 14 and plate aperture 24 may also be reversed. In this regard, since the locking element 14 opens at an angle between approximately 1 and 10 degrees when the fastener 16A is placed into the locking element 14, material can be removed from the edges of the locking element 14 to accommodate for the angular splay. If material is removed from the locking element 14, the edges of the aperture 24 should remain parallel to one another. In this way, after the constrained fastener 16A is placed into the locking element 14 within the plate 12, the locking element 14 will splay open such that its walls align with the planar segments 40A and 40B defining the aperture 24, thereby causing a more secure lock due to increased surface area contact.

Increased rigidity may also be accomplished by using other geometric forms that give edges similar to those described above for both the plate aperture 24 and the walls of the locking element 14. For example, other means of obtaining similar geometries may utilize ovals, ellipses, shapes that consist of a series of straight lines, concave curves, and/or convex curves linked together, polygons with rounded corners, and polygons with sharp corners.

The plate aperture 24 may also be tapered as it passes though the plate 12 to provide extra strength to the construct. By having a taper greater than 0 degrees in which the top of the aperture 24 is smaller than the bottom of the aperture 24, the same wedging concept is created when the fastener 16 is placed at extreme angles in the cephalad/caudal direction. Alternatively, the top of the aperture 24 may be larger than the bottom of the aperture or the top and bottom of the aperture 24 may be equal. In the same way, when the fastener 16 beings to sweep from its extreme angulation towards the center of the plate 12, the tapering of the aperture 24 stops the motion of the fastener/locking element assembly by forcing the locking element 14 deeper into the wedge created by the tapered geometry.

The locking concept discussed above may also be applied to the locking element 14, rather than to the plate aperture 24. In this case, an angle greater than 0 degrees may be applied to the faces 60A, 60B of the locking element 14 from which the tabs 62 originate. The larger distance from face 60A to face 60B across the opening 56 in the locking element 14 may be located near the top surface 90 of the locking element 14 and the smaller distance would be near the bottom surface 92. Alternatively, the larger surface may be near the bottom surface 92 or the top and bottom of the aperture 24 may be equal. In this way, when the fastener 16 is placed in the locking element 14 at an extreme cephalad/caudal angulation and the construct is placed under compressive load, the locking element 14 will act as a wedge. The wedge impedes the motion of the fastener and holds it fixed to the plate 12 when the fastener/locking element assembly tries to sweep towards the center of the plate 12. This increased rigidity may once again be accomplished by using other geometric forms that define features similar to the ones described above.

Both of the tapered options described above may be used independently or in combination to help increase the rigidity of the constrained fastener 16B construct. In certain applications, the plate 12 may utilize both the trapezoidal geometries and the tapered geometries as discussed above. Similarly, the taper options discussed above with respect to the locking element 14 may also be used in the combination to help increase the rigidity of the constrained fastener 16B construct. Finally, one tapered locking element option may be paired with the tapered plate hole.

While specific examples have been described in the specification and illustrated in the drawings, it will be understood by those skilled in the art that various changes may be made and equivalence may be substituted for elements thereof without departing from the scope of the present teachings as defined in the claims. Furthermore, the mixing and matching of features, elements and/or functions between various examples may be expressly contemplated herein so that one skilled in the art would appreciate from the present teachings that features, elements and/or functions of one example may be incorporated into another example as appropriate, unless described otherwise above. Moreover, many modifications may be made to adapt a particular situation or material to the present teachings without departing from the essential scope thereof. Therefore, it may be intended that the present teachings not be limited to the particular examples illustrated by the drawings and described in the specification as the best mode of presently contemplated for carrying out the present teachings but that the scope of the present disclosure will include any embodiments following within the foregoing description and any appended claims.

1. A bone plating system comprising:
a plate having a bone screw aperture, the bone screw aperture defined by a sidewall including first and second generally planar segments; and
a locking element coupled to the plate and at least partially positioned in the aperture, the locking element having an external geometry defining first and second generally planar surfaces and an internal geometry for receiving a head of a bone fastener and preventing the bone fastener from backing out of the plate, the locking element being expandable from a first state to a second state such that in the first state the locking element is permitted to articulate relative to the plate and in the second state the first and second generally planar surfaces of the locking element engage the first and second generally planar segments of the aperture sidewall, respectively, to prevent relative movement between the locking element and the plate.

2. The bone plating system of claim 1, wherein the first and second segment of the sidewall aperture are substantially parallel to one another.

3. The bone plating system of claim 1, wherein the locking element is coupled to the plate for relative movement in a first plane.

4. The bone plating system of claim 1, wherein the locking element is coupled to the plate for relative movement in a first plane and a second plane, the range of motion of the locking element relative to the plate in the first plane being substantially greater than the range of motion of the locking element relative to the plate in the second plane.

5. The bone plating system of claim 4, where the bone plating system is a spinal plating system, the first plane is the sagittal plane and the second plane is the transverse plane.

6. The bone plating system of claim 1, wherein the locking element includes at least a portion that is resiliently expandable for transitioning the locking element from the first state to the second state.

7. The bone plating system of claim 1, in combination with the bone fastener, the bone fastener being a fixed angle fastener having a head with a diameter sufficiently large to resiliently expand a portion of the locking element and transition the locking element from the first state to the second state upon seating in the locking element.

8. The bone plating system of claim 1, in combination with the bone fastener, the bone fastener being a variable angle fastener having a head with a diameter sufficiently small to retain relative articulation between the locking element and the plate upon seating in the locking element.

9. A bone plating system comprising:
a plate having a bone screw aperture, the bone screw aperture defined by a sidewall including first and second sidewall openings; and
a locking element coupled to the plate and at least partially positioned in the aperture, the locking element having an internal geometry for receiving a head of a bone fastener and preventing the bone fastener from backing out of the plate and an external geometry defining first and second tabs extending into the first and second sidewall openings, respectively, the first and second tabs and sidewall openings configured to normally allow articulation of the locking element relative to the plate in a first plane, at least one of the first and second tabs and at least one of the sidewall openings defining cooperating stop surfaces for limiting a range of articulation in the first plane.

10. The bone plating system of claim 9, wherein the first and second tabs and the sidewall openings are configured to normally allow articulation of the locking element relative to the plate in a second plane generally perpendicular to the first plane.

11. The bone plating system of claim 9, wherein the first and second tabs and the sidewall openings are configured to normally allow translation of the locking element relative to the plate in a plate plane of the plate.

12. The bone plating system of claim 9, wherein the locking element is coupled to the plate for relative movement in the first plane and a second plane, the range of motion of the locking element relative to the plate in the first plane being substantially greater than the range of motion of the locking element relative to the plate in the second plane.

13. The bone plating system of claim 9, wherein the at least one tab includes a first stop surface and the sidewall opening includes a second stop surface, the first and second stop surfaces spaced apart from one another when the locking element is in a neutral position and the first and second stop surfaces engaging one another when the locking element is rotated relative to the plate in the first plane through a predetermined range of articulation.

14. The bone plating system of claim 13, wherein the predetermined range of articulation is between approximately 20 degrees and approximately 35 degrees.

15. The bone plating system of claim 13, wherein the predetermined range of articulation is at least approximately 30 degrees.

16. The bone plating system of claim 9, wherein the locking element includes at least a portion that is resiliently expandable to transition the locking element from a first state to a second state, such that in the first state the locking element is permitted to articulate relative to the plate and in the second state relative movement between the locking element and the plate is prevented.

17. The bone plating system of claim 16, in combination with the bone fastener, the bone fastener being a fixed angle fastener having a head with a diameter sufficiently large to resiliently expand the portion of the locking element and transition the locking element from the first state to the second state upon seating in the locking element.

18. The bone plating system of claim 16, in combination with the bone fastener, the bone fastener being a variable angle fastener having a head with a diameter sufficiently small to retain relative articulation between the locking element and the plate upon seating in the locking element.

19. The bone plating system of claim 9, wherein the at least one tab and the corresponding sidewall opening have substantially equal heights such that articulation of the locking element relative to the plate in a second plane generally perpendicular to the first plane is substantially prevented.

20. A bone plating system comprising:
a plate having a bone screw aperture;
a constrained bone screw having a constrained head;
a semi-constrained bone screw having a semi-constrained head; and
a locking element at least partially positioned in the aperture and coupled to the plate for relative articulation within at least a first plane generally perpendicular to the plane of the plate, the locking element configured to interchangeably receive both the constrained bone screw and the semi-constrained bone screw such that the respective constrained and semi-constrained heads are captured relative to the locking element for articulation with the locking element and to prevent the respective bone fastener from backing out of the plate, the locking element expandable from a first state to a second state such that in the first state the locking element is permitted to articulate relative to the plate and in the second state relative movement between the locking element and the plate is prevented;
wherein the constrained head is configured to expand the locking element to the second state when captured by the locking element and the locking element remains in the first state upon capture of the semi-constrained head.

21. The bone plating system of claim 20, wherein the constrained head has a maximum diameter greater than a maximum diameter of the semi-constrained head.

22. The bone plating system of claim 20, wherein the locking element frictionally engages a sidewall of the bone screw aperture in the second state.

23. The bone plating system of claim 20, wherein the plate includes a longitudinal axis and the first plane is generally parallel to the longitudinal axis.

24. The bone plating system of claim 20, wherein the locking element is further coupled to the plate for relative movement in a second plane generally perpendicular to the plane of the plate, the range of motion of the locking element relative to the plate in the first plane being substantially greater than the range of motion of the locking element relative to the plate in the second plane.

25. A spinal plating system for securing a first vertebra and a second vertebra, the spinal plating system comprising:
a plate having first and second bone screw apertures for overlying the first vertebra and third and fourth bone screw apertures for overlying the second vertebra, at least one of the bone screw apertures is defined by a sidewall including first and second substantially planar segments, the first and second substantially planar segments including first and second sidewall openings, respectively; and
a locking element at least partially positioned in the one of the apertures, the locking element having an external geometry defining first and second substantially planar surfaces and first and second tabs extending from the first and second substantially planar surfaces, respectively, and extending into the first and second sidewall openings, respectively, the locking element further including an internal geometry for receiving a head of a bone fastener and preventing the bone fastener from backing out of the plate, the locking element being expandable from a first state to a second state such that in the first state the locking element is permitted to articulate relative to the plate and in the second state the first and second substantially planar surfaces of the locking element engage the first and second substantially planar segments of the aperture sidewall, respectively, to prevent relative movement between the locking element and the plate, at least one of the first and second tabs and at least one of the sidewall openings defining cooperating stop surfaces for limiting a range of articulation in the sagittal plane.

26. The bone plating system of claim 25, wherein the plate is a multi-level plate.

27. The spinal plating system of claim 25, further comprising a fixed angle fastener and a variable angle fastener interchangeably received within the locking element, the fixed angle fastener having a head with a diameter sufficiently large to resiliently expand a portion of the locking element and transition the locking element from the first state to the second state upon seating in the locking element, the variable angle fastener having a head with a diameter sufficiently small to retain relative articulation between the locking element and the plate upon seating in the locking element.

28. A bone plating system comprising:
a plate generally defining a plate plane having a bone screw aperture, the bone screw aperture defined by an aperture sidewall, the aperture sidewall having a first circumferential portion and a second circumferential portion, the second peripheral portion having an axis generally perpendicular to the plate plane, the first circumferential portion oriented relative to the axis at an angle of at least approximately 25 degrees;
wherein a bone screw is insertable into the aperture at an angle to the axis of at least approximately 25 degrees without interference from the aperture sidewall.

29. The bone plating system of claim 28, wherein the first circumferential portion is oriented relative to the axis at an angle of at least approximately 30 degrees.

30. The bone plating system of claim 28, wherein the first circumferential portion defines a portion of a cylinder.

31. The bone plating system of claim 28, further comprising a locking element at least partially positioned in the aperture and coupled to the plate for relative articulation within at least a first plane generally perpendicular to the plane of the plate, the first plane intersecting the first circumferential portion.
